(19) United States
(12) Patent Application Publication
(10) Pub. No.: US 2017/0052193 A1
(43) Pub. Date: Feb. 23, 2017

(54) FERTILITY AND PREGNANCY MONITORING DEVICE AND METHOD

(71) Applicant: Concepta Diagnostics Limited, York (GB)

(72) Inventors: Robert Porter, York (GB); Zhi Zhang, York (GB)

(73) Assignee: Concepta Diagnostics Limited, Yorkshire (GB)

(21) Appl. No.: 15/118,613

(22) PCT Filed: Feb. 13, 2015
(86) PCT No.: PCT/GB2015/050402

§ 371 Date: Aug. 12, 2016

(30) Foreign Application Priority Data
Feb. 14, 2014 (GB) 1402668.6

(51) Int. Cl. G01N 33/68; G06F 19/00; G01N 33/84; G01N 33/487; G01N 33/76; G01N 21/78

(52) U.S. Cl. CPC G01N 33/689; G01N 33/76; G01N 21/78; G01N 33/84; G01N 33/48792; G06F 19/3406; G01N 2800/368; G01N 2333/59; G01N 2021/7759

(57) ABSTRACT

A pregnancy monitoring device and method improve detection in the first trimester of the occurrence of ectopic pregnancy and the occurrence of multiple foetuses. The device includes a sample absorbing member and an analyser to analyse a plurality of sequential a biological liquid samples and computing means to determine an ovulation date and deviations in the measured quantity of hCG from predetermined values which are indicators of ectopic pregnancy or multiple pregnancy. The device and method enables early detection of ectopic pregnancy or multiple pregnancy.

There is disclosed a fertility and pregnancy monitoring device and method. In particular, there is disclosed a convenient and easy to use pregnancy monitoring device and method able to improve detection of the occurrence of ectopic pregnancy, miscarriage and the occurrence of multiple pregnancy in the first trimester.

### BACKGROUND

The first trimester is a well-known period of a pregnancy where complications occur. Many of these complications can be treated and resolved if detected early. In the UK there were over 700,000 live births with around 3,500 stillbirths at full term, and an estimated 117,000 pregnancies resulting in miscarriage or ectopic pregnancy. In Europe there were 5.2 million children born with an estimated 832,000 miscarriages or ectopic pregnancies. The incidence of problems in pregnancies in the first trimester is therefore high.

In the early stages of pregnancy, an egg is released from the ovaries into the Fallopian tubes, where it is fertilised by sperm.

If the pregnancy progresses normally, cilia within the Fallopian tubes push the fertilised egg along the tube and into the womb, where the egg implants itself into the womb's lining (endometrium) and develops into a baby.

In some situations, the cilia may not be able to move the egg to the womb and the pregnancy may develop in the fallopian tube. This is an ectopic pregnancy because the egg implants itself and develops outside of the womb.

Certain factors can increase the risk of having an ectopic pregnancy. Some diseases such as pelvic inflammatory disease (PID), often caused by chlamydia or gonorrhoea, may cause damage to the Fallopian tubes such as a blockage or narrowing of the tubes. Furthermore, it is difficult to identify a particular case of a woman who may be prone to ectopic pregnancy, because many of the diseases are caused by bacterial infections which often exhibit no noticeable symptoms and therefore a woman may be unaware that she is infected. However, the bacteria can cause inflammation of the Fallopian tubes, which is known as salpingitis. Salpingitis has been shown to lead to a four-fold increase in the risk of having an ectopic pregnancy.

Having a previous history of ectopic pregnancy also increases the risk of having a subsequent ectopic pregnancy compared to other women who have no history of ectopic pregnancy. Depending on the underlying factors, the risk of having another ectopic pregnancy is somewhere between 1 in 10 and 1 in 4.

In a few cases an ectopic pregnancy causes no noticeable symptoms and is only detected during routine pregnancy scanning. However, most women do have symptoms and these usually become apparent between week five and week 14 of pregnancy. The symptoms include abnormal vaginal bleeding and abdominal pain which can range from mild to severe.

If an ectopic pregnancy is detected at an early stage, medication can be used to stop the egg developing. The pregnancy tissue is then absorbed into the woman's body and no or little long term physical effect is experienced by the mother. Obviously, the emotional impact of having an ectopic pregnancy can be far reaching and often sufferers are offered counseling to help overcome the trauma.

Medication is not always needed, as in around half of cases of ectopic pregnancy the fertilised egg dies before it can grow larger.

However, if an ectopic pregnancy is left to develop, there is a risk that the fertilised egg continues to grow and cause the fallopian tube to split open (rupture), which can cause life-threatening internal bleeding. Advanced stage ectopic pregnancies require surgery to remove the egg, putting the mother under the additional risk of surgery and increasing the cost of care.

Another major concern during the first trimester is the risk of miscarriage. Whilst a small number of miscarriages are due to poor egg viability, the majority are due to hormonal, dietary or other forms of imbalance. Early identification of an abnormal pregnancy allows for further tests and the provision of a treatment regime to see if imbalances can be rectified and a healthy viable pregnancy established.

Other complications may occur during the first trimester including the possibility of a multiple pregnancy, when the mother is pregnant with two or more babies. Multiple pregnancies place the mother at risk to pregnancy related complications. If multiple pregnancy is detected in a woman very early in the first trimester, caregivers can adapt the support of the mother and improve environmental conditions to reduce the risks of miscarriage, anaemia, high blood pressure, pre-eclampsia, gestational diabetes, haemorrhage, and early labour.

In some instances, environmental factors can reduce the risk of ectopic pregnancy, potential miscarriage or multiple pregnancy and therefore, miscarriage and ectopic pregnancy can be avoided by improving environmental conditions. However, in such situations identifying the problem as early as possible is the key to a successful pregnancy.

Furthermore, drugs such as methotrexate can be used to prevent the Fallopian tubes splitting in the case of an ectopic pregnancy that continues to grow. Methotrexate may prevent the need for surgery if an ectopic pregnancy is identified early enough.

There is a need for a convenient and easy to use pregnancy monitoring device and method that can be used at home or at the doctor's surgery to improve detection of the occurrence of ectopic pregnancy, miscarriage and the occurrence of multiple pregnancy in the first trimester.

### BRIEF SUMMARY OF THE DISCLOSURE

In accordance with a first aspect there is provided a fertility and pregnancy monitoring device for detecting and monitoring hormones in a biological liquid sample comprising:

a sample holder configured to accept a sample absorbing member that has absorbed a portion of the biological liquid sample;

an analyser; and

a display means;

wherein the analyser is configured to analyse the sample absorbing member in the sample holder by measuring a parameter indicative of a quantity of luteinizing hormone (LH) present in the biological liquid sample and by measuring a parameter indicative of a quantity of a human chorionic gonadotropin hormone (hCG) in the biological liquid sample, the analyser being further provided with electronic computing means; and wherein the computing means is configured to calculate and record a plurality of measurements from a plurality of sequential sample absorbing members and to identify a predetermined increase in the measured quantity of LH in the biological liquid sample to determine an ovulation date and/or time while also identifying and recording the measured quantity of hCG in the biological liquid sample, where deviations in the measured quantity of hCG from predetermined values are determined to be indicators abnormal pregnancy and wherein an indication of the health of the pregnancy is displayed on the display means.

A benefit of the device is that the user accumulates a set of data within the device relating to LH and/or hCG hormone levels over a number of days and even multiple times a day. In this manner the device can determine the ovulation date, or depending upon the frequency of the samples, the device can determine an ovulation date and an ovulation time. The device monitors the measured quantity of LH and/or hCG in a biological sample and determines deviations from predetermined values. During a normal pregnancy (i.e. when the mother is carrying one baby) the measured quantity of hCG is expected to double in the two to three days immediately after conception. Surprisingly, the Applicant has found that deviations in the measured quantity of hCG are indicative of miscarriage, ectopic pregnancy or multiple pregnancy, and therefore the device can be used to identify miscarriage, ectopic pregnancy or multiple pregnancy early in pregnancy. Furthermore, the hCG hormone typically peaks after eight to eleven weeks of pregnancy and therefore the device may be used to determine whether the pregnancy is progressing normally.

If the measured quantity of hCG increases more rapidly, the woman may have a multiple pregnancy, and again, knowledge of multiple pregnancy early in the first trimester can lower the risks to the woman and the babies because the pregnant woman's medical support can be tailored to her individual needs.

The device is simple to use and portable and as such may be used in non-clinical environments. Presently there are no solutions for personal tracking of LH and hCG hormones in a non-clinical environment using a simple portable device. Combining the analysis of the LH and hCG hormones on a single device allows precise determination of whether the hCG levels are normal or abnormal. By identifying the increase in the measured quantity of LH and the measured quantities of hCG it is easy to rapidly identify whether a pregnancy is a problem pregnancy.

Furthermore, the device is configured to record the measured quantities of LH and hCG from a plurality of sequential samples and therefore the device can be used to map out the expected hormone levels over the course of several menstrual cycles. Some women may experience increased levels of LH that are not indicative of the date and time of ovulation. This is especially useful for users who find it difficult to conceive as it provides an indication of hormone levels over a number of cycles.

In some situations, increased levels of LH may be due to environmental reasons or other reasons such as stress. Continuing to measure and record the level of LH over a number of cycles enables the user to identify any phantom increases in LH, and the device can more accurately determine the true ovulation date and/or time.

Optionally, the device measures the parameter indicative of a quantity of LH present in the biological liquid sample over a number of menstrual cycles in order to identify the predetermined increase in LH indicating the ovulation date and/or time. The predetermined increase in LH is user specific and the level of a surge in LH for a particular user is dependent on the user.

Optionally, the device is configured to identify an egg fertilisation date and/or time. The egg fertilisation date and/or time is the date and/or time that the egg is fertilised. By increasing the number of biological samples sequentially measured for both LH and hCG levels, the egg fertilisation date and/or time may be identified.

Optionally, the indication of the health of the pregnancy includes information such as when the user should insert a sequential biological sample on a sample absorbing member, when the user should visit a health practitioner, and when a reading is deemed inaccurate.

A sequential sample absorbing member is a sample absorbing member having a sequential biological sample absorbed in the sample absorbing member. Sequential refers to taken in sequence in time. For example, a first biological sample taken by the user at 10 am on day one, and is followed by a second biological sample taken by the user at 10 am on day two. If more accurate forecasting is required, the user may increase the number of sequential biological samples taken and subsequently tested. A considerable benefit of the device is the ease of use by the user. Under normal pregnancy conditions, the user is able to continue providing biological samples harvested at home, and is able to easily use the device to monitor the health of the pregnancy.

Optionally, the analyser may analyse a number of measurable parameters such as colour, pH, transmission spectra, or absorption spectra of the sample absorbing member.

Optionally, the analyser may be an optical analyser able to identify changes in the colour of the sample absorbing member.

Optionally, the analyser may be a pH analyser able to identify the pH level of the sample absorbing member.

Optionally, the analyser may be an electrochemical analyser coupled with an ion selective electrode, able to identify a pH or salt concentration within the sample absorbing member or able to identify an electroactive label.

Optionally, the analyser may measure a concentration or a signal. Signal may refer to optical density or colour gradient or other optical measurement which may be converted to a concentration value.

Optionally, the electroactive label may be a metal particle label, a conducting polymer label or an enzyme label.

Optionally, the device may be provided with connection means to connect the device to an external device.

Optionally, the device may be provided with communication means to communicate with an external device.

Optionally, the biological sample may be selected from a mid-stream urine sample, a blood-based sample such as blood, plasma, or serum, an interstitial fluid sample, a saliva sample, or a gingival fluid sample.

Optionally, an alarm may be provided configured to indicate a number of different situations.

In accordance with a second aspect, there is provided a method for detecting and monitoring hormones in a biological liquid sample for identifying normal pregnancy, ectopic pregnancy, multiple pregnancy or an increased risk for miscarriage comprising the steps of:

obtaining a set of time-separated sequential biological liquid samples;

measuring a parameter indicative of a quantity of LH in the biological liquid samples;

determining the presence of a predetermined change in the quantity of LH and attributing an ovulation date corresponding to the date of the measurement;

measuring a parameter indicative of a quantity of the quantity of hCG in the sample;

determining the change in the quantity of measured quantity of hCG in each subsequent biological liquid sample and calculating the relative change in the measured hCG;

determining whether the relative change in measured quantity of hCG deviates from predetermined values relative to the ovulation date;

returning a healthy result if the measured hCG is within predetermined values; or returning a warning if the measured hCG deviates from predetermined values.

Optionally, the healthy result comprises information including any of: the measured quantity of LH, the measured quantity of hCG, the date and time of the test, and an amount of time between the time separated sequential tests.

Optionally, the warning comprises information including any of: the measured quantity of LH, the measured quantity of hCG, the date and time of the test, an amount of time between the time separated sequential tests, a request for a retest, and a recommendation to visit a healthcare practitioner.

Optionally, an ectopic/miscarriage result may be returned if the measured hCG is below the predetermined values relative to the ovulation date wherein the ectopic/miscarriage result is indicative of a potential ectopic pregnancy or a potential miscarriage.

Optionally, a multiple result may be returned if the measured hCG is above the predetermined values relative to the ovulation date wherein the multiple result is indicative of a potential multiple pregnancy.

Optionally the parameter indicative of the quantity of LH in the biological liquid sample may be monitored and analysed for approximately five to seven days after the ovulation date to indicate the health of the pregnancy.

In accordance with a third aspect, there is provided a kit of parts comprising:

a device according to the first aspect; and

a sample absorbing member.

In accordance with a fourth aspect, there is provided a use of a first sample absorbing member to measure a parameter indicative of the quantity of LH and the use of a second sample absorbing member to measure a parameter indicative of the quantity of hCG, wherein the first and second sample absorbing member are inserted into a pregnancy monitoring device of the first aspect.

### BRIEF DESCRIPTION OF THE DRAWINGS

Embodiments of the invention are further described hereinafter with reference to the accompanying drawings, in which:

FIG. 1 shows an embodiment of a fertility and pregnancy monitoring device;

FIG. 2 shows an expanded view of an embodiment of the fertility and pregnancy monitoring device;

FIG. 3 shows a graph showing the measured relative parameters indicating quantities of LH and hCG with respect to time for several women;

FIG. 4 shows an estimated hCG level versus time for 1000 samples;

FIG. 5 shows a probability of test error for a percentage deviation from the device obtaining a correct value;

FIG. 6 shows an example of a device.

### DETAILED DESCRIPTION

A normal pregnancy is a pregnancy where the woman is carrying one healthy foetus.

An ectopic pregnancy is a pregnancy where the egg implants itself and develops outside of the womb.

A multiple pregnancy is where the woman is carrying more than one healthy foetus.

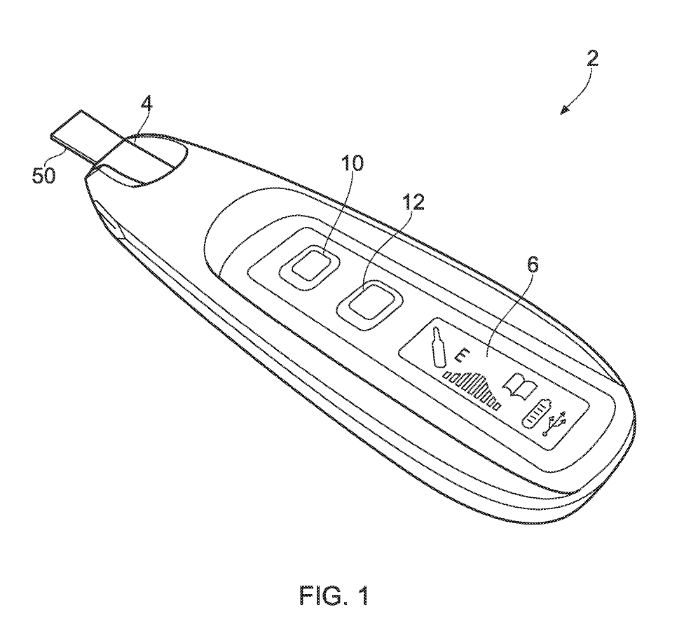

FIG. 1 shows the pregnancy monitoring device 2 including a sample holder 4 for holding a sample absorbing member (test strip) 50, an analyser, a display 6, and an electronic computer. The test strip absorbs a biological sample and the test strip is inserted into the sample holder. The biological sample may be obtained from a number of sources such as a urine sample taken from the mid-stream of a urination where the urine has been in the user's bladder for a number of hours. Other biological samples include a blood-based sample such as blood, plasma, or serum, an interstitial fluid sample, a saliva sample, or a gingival fluid sample. The test strip is designed to optimally absorb the chosen biological sample.

The analyser is an optical analyser comprising a photodiode and an organic light-emitting diode (OLED). A cradle is provided to support the test strip and hold it in alignment with the optical analyser ready for analysing. The analyser is configured to analyse the test strip and measures a parameter indicative of the amount of LH and hCG hormone in the test strip.

Alternative analysers may be used if the analyser is configured to measure the amount of LH or hCG in a specifically chosen biological sample. For example, the analyser may be a pH analyser able to identify the pH level of the sample in the sample absorbing member.

Alternatively, the analyser may be an electrochemical analyser coupled with an ion selective electrode, able to identify a pH or salt concentration. The analyser may be an electro-analytical analyser able to identify a change in electroactive label. The electroactive label may be embedded in the sample absorbing member and may be a metal particle label, a conducting polymer label or an enzyme label.

The device includes a display. The display is in communication with the computing means including electronic circuitry, internal memory or connection to external memory. The computing means is configured to calculate and record a plurality of measurements from a plurality of sequential urine samples. The computing means is able to identify a predetermined increase in the measured quantity of LH and to determine an ovulation date and or time from the increase in measured quantity of LH.

The display displays the results including the ovulation date and/or time. The display shows the present time and date and also shows other information including the battery power remaining, the number of days since the last menstruation, the number of days since ovulation, a pregnant or not pregnant indicator and the health of the pregnancy. On the screen there may also be an icon or other means of information that shows connectivity between the device and an external computing device by wireless or wired connection. There may also be situation where the device could give an indication that the user should go and see her health care provider. The device may also indicate the occurrence of an abnormal middle cycle menstrual bleed event during pregnancy or prior to conception.

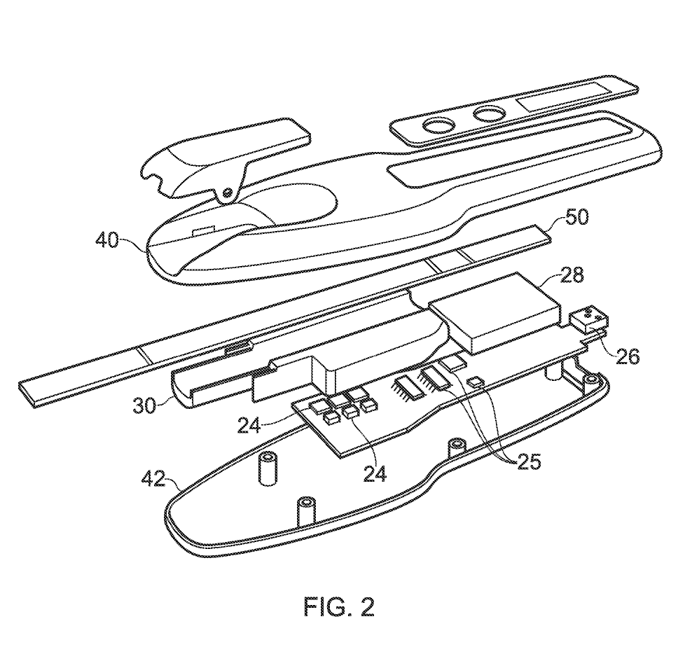

FIG. 2 shows an expanded view of an embodiment of the device. The outer housing includes an upper housing 40 and a lower housing 42. There are connecting members that connect the upper housing 40 to the lower house 42. The upper housing includes the display 6 and control buttons 10, 12. Inside the housing is the cradle 30 for supporting a test strip 50 in predefined position so that the analyser can analyse the test strip 50. The optical analyser includes photodiodes and OLEDs 24. The cradle supports the test strip in position so that the OLEDs sufficiently illuminate the test strip so that the photodiodes can measure the signals (reflection, absorption or transmission spectra) from the test strip. The signals are then processed using the processor 25 and the results are displayed on the display 6. The results may also be transmitted to an external processor or other computing device wirelessly, or via a USB connector 26.

The device simultaneously measures and records a measure of the quantity of LH and hCG from the biological sample absorbed on the test strip. The device determines the ovulation date from the measurements of LH and the device determines the health of the pregnancy from the measured quantity of hCG and the measured quantities of LH. If the measured quantity of hCG (or LH) deviates from predetermined values, the results may indicate the occurrence of ectopic pregnancy, potential miscarriage or multiple pregnancy. Deviations in the measured quantity of hCG (or LH) can be displayed on the display means.

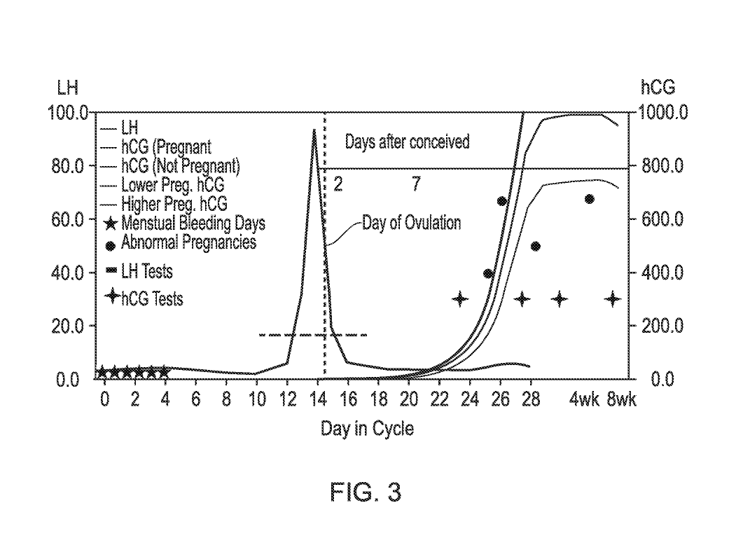

FIG. 3 is an illustration showing the measured parameters indicating the quantities of LH and hCG in a biological sample with respect to time in days or weeks.

At the beginning of a menstrual cycle (i.e. the beginning of the menstrual cycle is the first few days of the cycle which are the menstrual bleeding days) the measured level of LH is low. Although LH may fluctuate or rise within the first 14 days of the cycle, some fluctuations can be attributed to environmental conditions or even stress. However, a surge in the measured level of LH around day 12 to day 18 (typically around 10 to 12 days after the end of the last menstrual bleed) is indicative of the day of ovulation. The date and time of ovulation is the date and time immediately after the LH surge, just as the LH levels begin to drop as shown in FIG. 3.

The method for detecting and monitoring hormones in a biological liquid sample includes firstly, obtaining a set of time-separated sequential biological liquid samples. The samples are collected using a sample absorbent member such as a test strip. A parameter (colour, pH, photoluminescence for example) indicative of a quantity of LH in the test strip is measured with the measurements starting after five to six days from the day of the end of the menstrual bleeding days. The time-separated biological samples are analysed using an LH hormone test strip, which has a biological liquid sample absorbed within it. The test strips are analysed for LH every day (every day being the minimum number of samples required) for the next 7 days to detect the LH surge and thus the ovulation date and time, which is typically around 10-12 days after the last menstrual bleed. The LH surge is determined by measuring the presence of a predetermined change in the quantity of LH and attributing an ovulation date corresponding to the date of the measurement.

The test strips are analysed for LH content every day for the next 5 to 10 days after the predicted ovulation date to check that the measured LH surge is indicative of the ovulation date. If the measured LH continues to drop off, a phantom ovulation may have occurred. In this situation, a second LH peak is expected after the first, which represents the true ovulation LH surge.

Once the ovulation date has been determined, an hCG strip which has a biological liquid sample absorbed within it is analysed to determine if a rise in the pregnancy hormone (hCG) is occurring. The hCG strips are analysed every 2 to 5 days to determine the change in the quantity of measured hCG in each subsequent biological liquid sample and the relative change in the measured hCG is calculated. The relative change in the measured quantity of hCG is compared with predetermined values relative to the ovulation date. If the measured quantity is within predetermined values then a healthy result is returned. If the measured quantity deviates from predetermined values, a warning is returned.

The rate of testing for LH and for hCG levels varies depending on the users be necessary for high risk users. LH levels may be measured multiple times a day in order to accurately identify the date and time of ovulation.

Furthermore, the rate of testing of hCG levels may be increased to more accurately identify the egg fertilisation date and/or time.

The hCG strips may be monitored for several weeks with fresh sample every 2-5 days to track the HCG, to see if the HCG is doubling or tripling approximately every 2 days. This is done for six weeks after the estimated day of ovulation or approximately 8 weeks from the last menstrual period.

An example of expected levels of hCG in blood is shown below:

| Duration from LMP* | | Range of hCG (mIU/ml) |
|---|---|---|
| 3 | Weeks | 5 to 50 |
| 4 | Weeks | 5 to 426 |
| 5 | Weeks | 18 to 7,340 |
| 6 | Weeks | 1,080 to 56,500 |
| 7 to 8 | Weeks | 7,650 to 229,000 |
| 9 to 12 | Weeks | 25,700 to 288,000 |

*LMP stands for last menstrual period.
Source: H. Murkoff and S. Mazel, What to Expect When You're Expecting, NewYork: Workman Publishing; 2008: 141.
The expected levels in the mid-stream of the first urine sample of the morning would be much lower than the blood values but would track the same pattern.

Combining the measurement of LH (i.e. the fertility hormone) and hCG (i.e. the pregnancy hormone) into one correlated result on the same device and then tracking the hCG rise on the same monitor improves early identification of ectopic pregnancy, potential miscarriage or multiple pregnancy.

By measuring the fertility hormone LH (i.e. the hormone that stimulates egg release and therefore the most fertile phase), the date of fertilisation and the date of ovulation can be precisely determined. Whilst the fertile period can be between 2-6 days, the time when the egg can be fertilised by the sperm is a much smaller period from hours to a couple of days.

Therefore knowing the precise time of the egg release and the date of copulation it can therefore allow for the accurate prediction of egg fertilisation. The fertile period is larger because the female hormones produce an environment where the sperm can be stable for a longer period, before the egg is released. The vertical line on FIG. 3 around day 15 is therefore the date and time when fertilisation of the egg can occur and therefore the hCG hormone tracking will start from this period.

The hCG hormone typically doubles every 2-3 days and peaks after 8-11 weeks of pregnancy. Problem pregnancies such as ectopic and miscarriage have a much flatter hCG rise or will rise normally or have a sudden drop in hCG. By determining the ovulation date determined from the LH measurements, and measuring hCG from that point on the same device a problem pregnancy can be identified quickly and precisely.

In certain circumstances, different types of sample absorbing members (test strips) may be used. The LH and hCG analysis may be carried out using an identical test strip or different test strips, each adapted to test a single hormone (either LH of hCG). The user would then be informed which type of test strip to use, and asked to insert each test strip after the first had been analysed.

The device provides an early identification means of potential issues that may occur throughout pregnancy. A deviation from normal levels of hCG during the progression of a pregnancy alerts the user to start a triage process to verify any potential condition the user may be experiencing. Normal levels of hCG are the same as predetermined values, expected hCG levels or predicted hCG projections.

hCG levels can fluctuate in early pregnancy. However, by using the measured LH surge as the starting point in time for measuring the hCG levels, the device can identify heightened risk of potential conditions far earlier than any known device or method because the device is able to accurately measure the LH surge and start hCG measurements and compare the measured values with expected hCG levels for normal pregnancies.

In one example, the device is configured so that if the projection of the hCG is within 20% of the expected hCG levels for normal pregnancies, the measurement of hCG level is indicative of healthy pregnancy. The device would return a healthy result.

The healthy result comprises information including any of: the measured quantity of LH, the measured quantity of hCG, the date and time of the test, and an amount of time between the time separated sequential tests.

In one example, the device is configured so that if the projection of the hCG is 20% lower than expected hCG levels for normal pregnancies, the measurement of hCG level is potentially indicative of an ectopic pregnancy. The device would return a warning to recommend the user to seek medical attention, or to carry out a further test, either using the same biological sample, or a new biological sample. The warning result comprises information including any of: the measured quantity of LH, the measured quantity of hCG, the date and time of the test, an amount of time between the time separated sequential tests, a request for a retest, and a recommendation to visit a healthcare practitioner.

In certain situations, the user may be able to change environmental conditions improve the viability of the pregnancy, due to early diagnosis and identification.

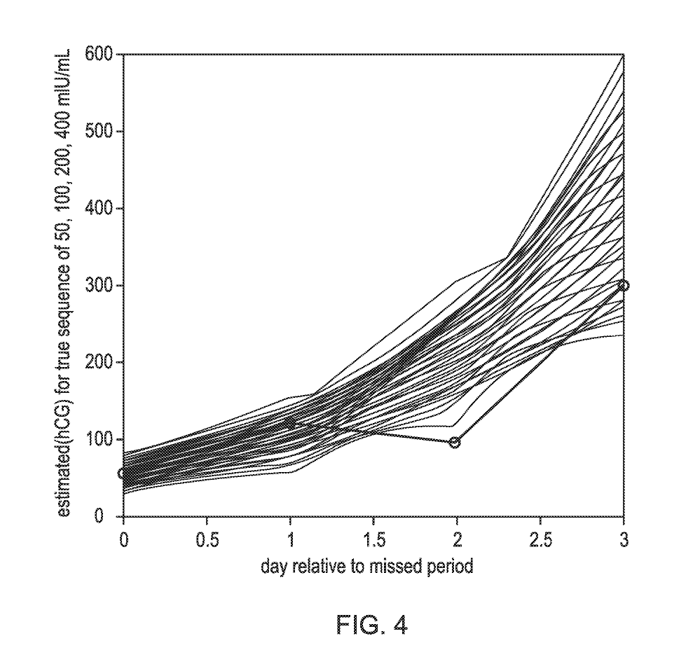

FIG. 4 shows the estimated hCG level versus time for 1000 samples. One sample (highlighted with a heavier black line) drops below the normal expected hCG level at day two, but returns back to the normal expected hCG level on day three, demonstrating that the measurement taken on day two outlier. Therefore, the device and method are configured to take into account the possibility of an outlier. In one example, the device may prompt the user to retest the sample. In another example, the device may prompt the user to provide a new sample.

In an example, the device is configured so that if the measured hCG level drops more than 20% below the normal expected hCG level, the device will display a request for a further sample to be tested after 24 hours. The second measurement is then used to identify whether the first measurement was an anomaly. If the subsequent measured hCG level remains more than 20% below the normal expected hCG level, the device will display a warning. If the hCG returns back to normal on the second day a healthy result. If the level remains more than 20% below the normal expected hCG level or drops further then a warning will be displayed on the device.

If the measured hCG level dramatically drops in one measurement of a biological sample by more than 60% of the normal expected hCG level shown in the table, a warning would be displayed on the device. This would raise an automatic system to ask the woman to go to go to the hospital for further test. If the woman has retained the urine sample a retest could be performed to see if it is a diagnostic error.

The device may be programmed so that a healthy result is displayed when the measured hCG level is within +/−2%, +/−5%, +/−7.5%, +/−10%, +/−15%, +/−20%, +/−25%, +/−30% or +/−40%. The programmed fluctuation percentage relative to the normal expected hCG level may depend on the user and the medical history of the user. High risk users may use less tolerant controls in order to obtain a healthy result so that a smaller fluctuation in the measured hCG level indicates that the user seek further testing or medical attention.

Furthermore, the device is able to collate date over extended periods of time. In some instances, the device is able to collate data over different pregnancies in order to identify the user's normal progression of hCG levels.

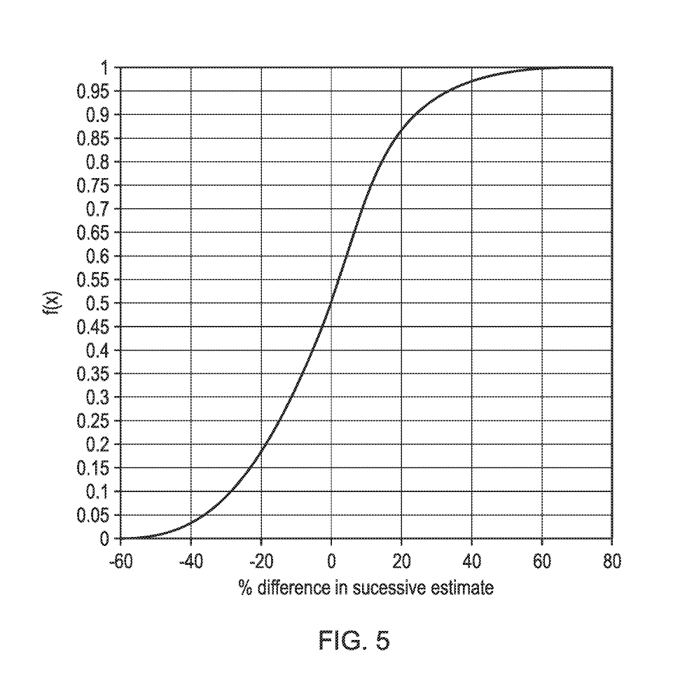

FIG. 5 shows the probability of test error for a percentage deviation from the device obtaining a correct value. The simulation shows that a 60% drop in predicted successive measurements and demonstrates that testing of hCG levels over a number of days is useful for improving accurate measurements.

In some examples, the device is adapted for use in detecting concentration markers in biological samples. Concentration markers such as creatine may be used to improve the accuracy of the detection of hCG levels in urine samples. The testing procedure would be once every two days within the first few weeks after the first missed period, this can be increased to 8 weeks if the user so requires.

The device is programmable so that the testing schedule is variable depending on a number of factors. For example, the testing frequency is reduced after the first eight weeks of pregnancy. The frequency of tests may reduce to a weekly test.

Molar pregnancy is thought to be caused by a problem with the genetic information of an egg or sperm. There are two types of molar pregnancy: complete and partial. Complete molar pregnancy occurs when an egg with no genetic information is fertilized by a sperm. It does not develop into a foetus but continues to grow as a lump of abnormal tissue that looks a bit like a cluster of grapes and can fill the uterus. Partial molar pregnancy occurs when an egg is fertilized by two sperm. The placenta becomes the molar growth. Any foetal tissue that forms is likely to have severe defects.

Molar pregnancy can progress to persistent trophoblastic disease (PTD) where the trophoblasts have invaded the maternal host and continue to proliferate even after evacuation of the molar pregnancy. Because of the highly proliferative nature of molar pregnancy, hCG levels may reach very high levels and rise more rapidly than normal pregnancies. Molar pregnancy can be associated with fertility treatment and there are cases of successful management of concurrent molar with a normal foetus through to delivery.

Molar pregnancy can be differentiated by hCG hyperglycosylation level. The device may be adapted to detect the hCG hyperglycosylation level in order to detect molar pregnancy.

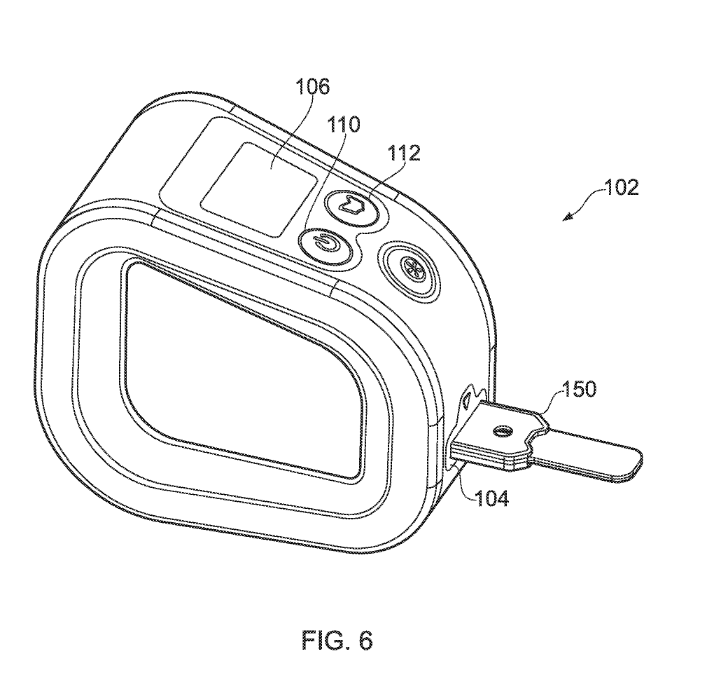

FIG. 6 shows an alternative design of device 102 including a sample holder 104 for holding a sample absorbing member 150, an analyser, a display 106, and an electronic computer having a number of controls 110, 112, and a display 106. The benefit of the alternative design is that it makes it easier for the user to hold and operate.

It will be clear to a person skilled in the art that features described in relation to any of the embodiments described above can be applicable interchangeably between the different embodiments. The embodiments described above are examples to illustrate various features of the invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments.

The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A fertility and pregnancy monitoring device, comprising:
a sample holder configured to accept a sample absorbing member that has absorbed a portion of a biological liquid sample;
an analyzer; and
a display means;
wherein the analyzer is configured to analyze the sample absorbing member in the sample holder by measuring a parameter indicative of a quantity of luteinizing hormone (LH) present in the biological liquid sample and by measuring a parameter indicative of a quantity of a human chorionic gonadotropin hormone (hCG) in the biological liquid sample, the analyzer being further provided with electronic computing means; and
wherein the computing means is configured to calculate and record a plurality of measurements from a plurality of sequential sample absorbing members and to identify a predetermined increase in the measured quantity of LH in the biological liquid sample to determine an ovulation date while also identifying and recording the measured quantity of hCG in the biological liquid sample, where deviations in the measured quantity of hCG from predetermined values are determined to be indicators abnormal pregnancy and wherein an indication of the health of the pregnancy is displayed on the display means.

2. A fertility and pregnancy monitoring device according to claim 1, wherein the analyzer is configured to analyze a number of measurable parameters such as colour, pH, transmission spectra, or absorption spectra of the sample absorbing member or the biological fluid sample.

3. A fertility and pregnancy monitoring device according to claim 1, wherein the analyzer is an optical analyzer that identifies changes in a colour of the sample absorbing member or the biological fluid sample.

4. A fertility and pregnancy monitoring device according to claim 1, wherein the analyzer is a pH analyzer that identifies the pH level of the sample absorbing member or the biological fluid sample.

5. A fertility and pregnancy monitoring device according to claim 1, wherein, the analyzer is an electrochemical analyzer coupled with an ion selective electrode to identify a pH or salt concentration of the sample absorbing member or the biological fluid sample, or able to identify an electroactive label within the sample absorbing member.

6. A fertility and pregnancy monitoring device according to claim 5, wherein, the electroactive label is a metal particle label, a conducting polymer label or an enzyme label.

7. A fertility and pregnancy monitoring device according to claim 1, further comprising connection means to connect the device to an external device.

8. A fertility and pregnancy monitoring device according to claim 1, further comprising communication means to communicate wirelessly with an external device.

9. A fertility and pregnancy monitoring device according to claim 1, wherein the sample absorbing member is configured to absorb a biological sample selected from a mid-stream urine sample, a blood based sample such as blood plasma or serum, an interstitial fluid sample, a saliva sample, or a gingival fluid sample.

10. A fertility and pregnancy monitoring device according to claim 1, further comprising an alarm configured to indicate a number of predetermined thresholds.

11. A fertility and pregnancy monitoring device according to claim 1, wherein the device measures the parameter indicative of a quantity of LH present in the biological liquid sample over a number of menstrual cycles to identify the predetermined increase in LH indicating the ovulation date and/or time.

12. A fertility and pregnancy monitoring device according to claim 1, wherein the device is configured to identify an egg fertilization date and/or time.

13. A fertility and pregnancy monitoring device according to claim 1, wherein the indication of the health of the pregnancy includes information chosen from when the user should insert a sequential biological sample on a sample absorbing member, when the user should visit a health practitioner, and when a reading is deemed inaccurate.

14. A method comprising the steps of:
obtaining from a patient a set of time-separated sequential biological liquid samples;
measuring with an analyzer a parameter indicative of a quantity of luteinizing hormone (LH) in the biological liquid samples;
determining by a processor an ovulation date for the patient based on the presence of a predetermined change in the quantity of LH in the biological liquid samples;
measuring with the analyzer a parameter indicative of a quantity of the quantity of human chorionic gonadotropin hormone (hCG) in the sample;
determining by the processor the change in the quantity of measured quantity of hCG in each subsequent biological liquid sample and calculating the relative change in the measured hCG;
determining by the processor whether the relative change in measured quantity of hCG deviates from predetermined values relative to the ovulation date;
returning a healthy result for display on a graphical user interface if the measured hCG is within predetermined values to identify for the patient a normal pregnancy; or
returning a warning for display on the graphical user interface if the measured hCG deviates from predetermined values to identify for the patient one of an ectopic pregnancy, a multiple pregnancy, or an increased risk for miscarriage.

15. A method according to claim 14, wherein an ectopic/miscarriage result may be returned if the measured hCG is below the predetermined values relative to the ovulation date wherein the ectopic/miscarriage result is indicative of a potential ectopic pregnancy or a potential miscarriage.

16. A method according to claim 14, wherein a multiple result may be returned if the measured hCG is above the predetermined values relative to the ovulation date wherein the multiple result is indicative of a potential multiple pregnancy.

17. A method according to claim 14, wherein the parameter indicative of the quantity of LH in the biological liquid sample may be monitored and analyzed for approximately five to seven days after the ovulation date to indicate the health of the pregnancy.

18. A method according to claim 14, wherein the healthy result comprises information including any of: the measured quantity of LH, the measured quantity of hCG, the date and time of the test, and an amount of time between the time separated sequential tests.

19. A method according to claim 14, wherein warning comprises information including any of: the measured quantity of LH, the measured quantity of hCG, the date and time of the test, an amount of time between the time separated sequential tests, a request for a retest, and a recommendation to visit a healthcare practitioner.

20. A kit of parts comprising:
a device according to claim 1; and
a sample absorbing member.

21. A fertility and pregnancy monitoring device according to claim 1, wherein a first sample absorbing member is inserted into the device to measure a parameter indicative of the quantity of LH and a second sample absorbing member is inserted into the device to measure a parameter indicative of the quantity of hCG.

22. A fertility and pregnancy monitoring device, comprising:
a sample holder configured to accept a sample absorbing member containing a biological liquid sample;
a graphic user interface configured for use by a patient;
an analyzer configured to measure, for each of a plurality of sequential sample absorbing members, a quantity of luteinizing hormone (LH) and a quantity of human chorionic gonadotropin hormone (hCG) present in the biological liquid sample; and
a processor configured to:
determine an ovulation date of the patient based on the respective measured quantities of LH in each of the plurality of sequential sample absorbing members;
compare the measured quantity of hCG in each of the plurality of sequential sample absorbing members to each of a respective predetermined hCG value and the ovulation date of the patient;
determine, for each comparison, a health status of a pregnancy of the patient; and
cause the graphic user interface to display at least one of an indication of the health status and an instruction for the patient to seek medical attention.
